(19) United States
(12) Patent Application Publication
(10) Pub. No.: US 2014/0163245 A1
(43) Pub. Date: Jun. 12, 2014

(54) PROCESS FOR PRODUCING FURFURAL FROM BLACK LIQUOR

(71) Applicants: Michael A. Lake, Mt. Pleasant, SC (US); John C. Blackburn, Easley, SC (US)

(72) Inventors: Michael A. Lake, Mt. Pleasant, SC (US); John C. Blackburn, Easley, SC (US)

(21) Appl. No.: 14/119,517

(22) PCT Filed: Apr. 26, 2012
(86) PCT No.: PCT/US12/35159

§ 371 Date: Nov. 22, 2013

Related U.S. Application Data
(60) Provisional application No. 61/490,249, filed on May 26, 2011.

(51) Int. Cl. C07D 307/50

(52) U.S. Cl. CPC C07D 307/50
USPC 549/489

(57) ABSTRACT

A process for making furfural using papermaking black liquor from the kraft pulping process as a feed material. First, the lignin is removed from the black liquor by carbonizing the black liquor to a pH below pH 10 to insolubilize the lignin, neutralize NaOH and other inorganic components of the black liquor. The next step is to treat the carbonated black liquor that contains the hemicellulose to remove the high molecular weight components. In a preferred embodiment the treatment uses multiple sequential steps. The first step of the treatment is to use ultrafiltration, centrifugation or dissolved-air floatation to separate the high molecular weight components. The second filtration is to pass the hemicellulose containing black liquor stream through a nanofilter to remove low molecular weight components. The conversion of xylans in the hemicellulose-containing mixture to furfural is accomplished using a catalytic process. The xylans are converted to pentose sugars and then converted to furfural. The furfural is formed at a low concentration and then further concentrated.

### CROSS-REFERENCE TO RELATED APPLICATIONS

The contents of Provisional Application U.S. Ser. No. 61/490,249 filed May 26, 2011 on which the present application is based and benefits claimed under 35 U.S.C. §119(e), is herein incorporated by reference.

### BACKGROUND OF THE INVENTION

1. Field of the Invention

The present invention relates to a process for producing furfural from black liquor. More particularly, this invention is to processes for the chemical conversion of the xylan fraction of the hemicelluloses in black liquor to furfural.

2. Description of the Prior Art

Black liquor is the spent cooking liquor from the kraft pulping process when digesting pulpwood into paper pulp. Black liquor contains a range of organic components, including lignin, hemicelluloses, and tall oil, as well as inorganic components. Hemicelluloses are heterogeneous polymers of pentoses, hexoses and sugars.

Furfural, furan-2-carboxyaldehyde, is the aldehyde of pyromucic acid. Furfural is prepared commercially by dehydration of pentose sugars:

C₅H₁₀O₅→C₅H₄O₂+3 H₂O

The major use of furfural is as a feedstock for furfuryl alcohol production, most of which is used in condensation reactions with formaldehyde, phenol, acetone or urea to yield resin with excellent thermosetting properties and extreme physical strength.

A process developed in the 1920's for manufacturing furfural, that disclosed in U.S. Pat. No. 1,919,877 by Brownlee, used oat hulls as a raw material and adds dilute acid to the oat hull in the digester and then passes steam continuously through the digester to produce furfural at a low concentration. However, today, commercial production of furfural has moved offshore from the US, primarily due to economics but also because of environmental pressure since the manufacturing creates acidic waste streams with very high BOD levels. Currently furfural is produced from corn cobs gathered at small farm-scale operations in China and processed at a central location close to a source of residual agricultural waste.

Furfural may be synthesized from C₅-hemicellulose containing agricultural wastes, such as corn cobs, cotton seed, oat, rice hulls and bagasse, using acid-catalyzed reactions that involve the hydration of polysaccharides (pentosans or xylans) into sugars (pentoses or xylose), which then undergo cyclodehydration to form furfural. The acid catalyzed hydration or depolymerization reactions are rapid in comparison to the latter dehydration reactions, but both occur readily under mild operating conditions. Dilute mineral acids (e.g., 3 wt % sulfuric acid solutions) are used to catalyze the hydration and dehydrocyclization reactions, but these processes are plagued with unwanted byproduct streams containing spent acid, which must be recycled or neutralized and dumped, and solids that are dumped or used as low grade fuel for onsite power boilers.

Significant advances to processes for the production of furfural have been achieved when traditional homogeneous mineral acid catalysts have been replaced with solid acid catalysts, which are more easily separated from the reaction mixture and reused. Of particular note, H-form zeolites, heteropolyacids, and sulfated metal oxides (e.g., sulfated zirconia) have shown promise as solid catalysts for these processes. These catalysts are robust, relatively inexpensive, and significantly reduce the amount of environmental waste generated. However, despite such advances there remains a need for a cost-efficient source of furfural.

### SUMMARY OF THE INVENTION

It is therefore the general object of the present invention to provide a process that uses an existing raw material supply, a by-product stream from pulp and paper mills, as the starting material to produce furfural.

Another object of the present invention is to provide a cost-effective source of furfural from which adhesive resins can be manufactured.

Yet another object of he present invention is to provide an environmentally-clean process, since the byproducts would be combined with black liquor within the papermaking system and burned for fuel value.

The present invention provides a process for making furfural using papermaking black liquor of the kraft pulping process as a feed material. The first step of the multi-step process is to remove lignin from said black liquor by carbonizing the black liquor to a pH <10 to insolubilize the lignin, neutralize NaOH and other inorganic components of the black liquor. The remaining organics are hemicelluloses which are themselves precursors to the industrial chemicals of this invention. Because the lignin is removed from the black liquor, the chemical reactivity is improved as well as concentrating the hemicellulose fraction of the black liquor.

The next step of the process is to treat the carbonated black liquor containing the hemicellulose fraction via filtration or centrifugation or dissolved-air flotation. In a preferred embodiment, the filtration step uses multiple sequential membrane separations. The first filtration may be ultrafiltration using a membrane process to reject the high MW material since most of the high molecular weight (MW) materials are solid at pH<10 and at ambient temperature. The second filtration passes the hemicellulose-containing black liquor mixture through a nanofilter to remove dissolved solids to remove inorganic salts and concentrate the remaining hemicellulose-containing mixture. The permeate from these separations, rich in Na⁺, is typically returned to the host papermaker.

The conversion of xylans in the hemicellulose-containing mixture to furfural is accomplished using a catalytic process. The xylans are converted to pentose sugars and then converted to furfural. Larger pore catalysts which may be used include, for example, amorphous sulfated zirconia or tungstosilicic acid. Smaller pore catalyst include, for example, zeolites, zeolite acid catalysts and mesoporous acid catalyst.

Once the furfural is formed it is generally at a low concentration and purification may be by way of extraction, distillation or come combination of both.

Furfural is used as a resin component as well as a precursor to furfuryl alcohol. Other uses for furfural include, for example, it use in refining lubricating oil.

### BRIEF DESCRIPTION OF THE DRAWINGS

Having described the invention in general terms, reference will now be made to the accompanying drawings, which are not necessarily drawn to scale, and wherein:

FIG. 1 is a diagram of the process of this invention for making furfural from black liquor.

### DESCRIPTION OF THE PREFERRED EMBODIMENT(S)

The present invention now will be described more fully hereinafter with reference to the accompanying drawing, in which a preferred embodiment of the invention is shown. This invention may, however, be embodied in many different forms and should not be construed as limited to the embodiments set forth herein; rather these embodiments are provided so that this disclosure will be through and complete and will fully convey the scope of the invention to those skilled in the art.

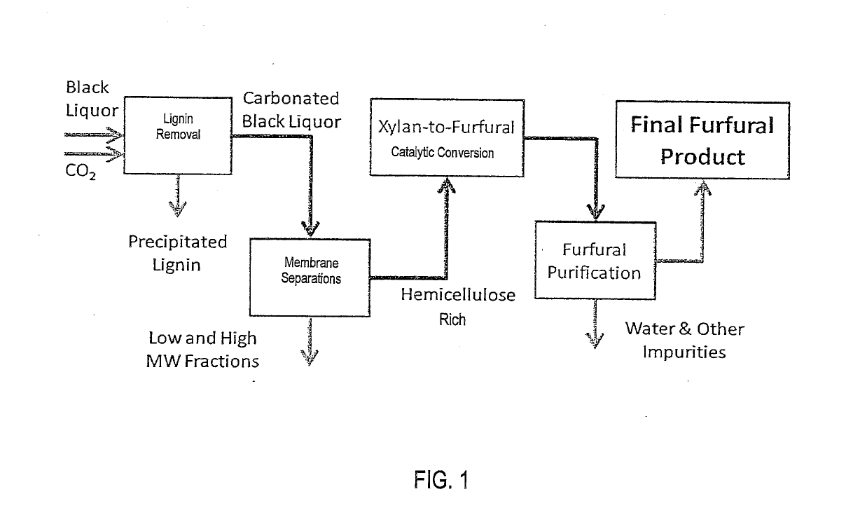

Referring now to FIG. 1, there is shown the steps of the process of this invention for making furfural using papermaking black liquor as a feed material using four separate unit operations: lignin precipitation and removal using carbonization of the black liquor, separation to concentrate the hemicellulose-containing mixture, chemical conversion of the xylan fraction of the hemicellulose-containing mixture to furfural, and purification to produce a concentrated furfural.

The present invention provides processes for producing furfural from black liquor from a paper making process by separation of the lignins from the black liquor. The soluble lignin at a pH between 12 and 14 is precipitated by introducing the black liquor, which may be under pressure, into an absorption column and treating the black liquor countercurrently with carbon dioxide (CO₂), to form NaHCO₃. The column may operate at a nominal pressure of 150 psig and a temperature between about 60° C. and 150° C., preferably about 110° C. to 130° C. In the column, the pH is lowered to below pH 9, preferably to between about pH 8.5 to pH 9.5, to partially neutralize the NaOH and other basic components within the black liquor. The carbon dioxide also converts much of the sodium (and other metals associated with the phenolic and carboxylic groups on the lignin molecules other forms including to the hydrogen form, causing the lignin to become insoluble and separate from the black liquor. The lignin is then recovered or returned to the recovery process of the host paper mill. This separation serves two purposes: (1) separation of the lignin makes the subsequent separations and conversions much less susceptible to fouling, and (2) reducing the pH will increase the membrane life of filters and allows a wider selection of filter membranes due to the less severe pH. The remaining organics are mostly hemicelluloses which are themselves precursors to the industrial chemicals of this invention. Because the lignin is removed from the black liquor, the chemical reactivity is improved as well as concentrating the hemicelluloses fraction of the black liquor. The concentration of hemicellulose in carbonated black liquor may be as high as 50% depending upon whether the black liquor is pine black liquor or hardwood black liquor.

The carbonated black liquor that contains the hemicellulose fraction is treated to concentrate the hemicelluloses in an aqueous solution and remove components such as sodium hydroxide and other salts that may interfere with subsequent processing steps. Although the treatment step may be accomplished in a single step when using filtration, preferably, the carbonated black liquor is subjected to two sequential membrane separations. First, an ultrafiltration step with a tubular membrane is used to remove the large (>1000 MW) organic fractions remaining in the black liquor. It is preferred that these membranes have a molecular weight (MW) cut-off of 1500-2000 which rejects as well the suspended solids. PCI membranes (from Membrane Specialists, LLC) have been shown to be effective in separating the high MW hemicellulose fraction. Other types of membranes that may be used include ceramic membranes form Ceramatec (Golden, Colo.). The hemicellulose separations are much cleaner when the high MW lignin has been removed. The breadth of commercial membranes available is much broader when exposed to carbonated black liquor at pH 10 rather than the normal black liquor at pH >13. The MW separation should be done at the temperature of the upstream process which in the operation described above is from about 110° C. to about 150° C. (preferably in the range of about 60° C. to about 130° C.), but it should be understood that this temperature will vary depending upon the particular upstream process used. In addition to ultrafiltration, centrifugation or dissolved-air flotation may be used to remove the high MW materials. The high MW lignin fractions have a high fuel value and are typically returned to the host papermaker.

Once the large organic fractions have been removed the hemicellulose-containing mixture is further filtered using a nanofilter to remove dissolved inorganic salts. Nanofilters pass monovalent ions and some multivalent ions (depending upon the pH) while retaining molecules with MW above a specified MW cut-off. It is preferred that the nanofilter have a MW cut-off of 150-500 MW in a spiral-wound configuration. Membrane Specialists, LLC and Koch Membranes are among the suppliers of nanofiltration membranes. The hemicellulose/xylan-containing mixture from the treatment step has a concentration in the range of about 5% to 40%.

As shown in FIG. 1, the next step is to convert the xylans to pentose sugars then convert the pentose sugars to furfural. Two separate catalyst materials are used; thus, optionally, two separate reactors can be used for the overall process. Larger pore catalyst materials, such as amorphous sulfated zirconia or tungstosilicic acid, are more effective at degrading the polymeric xylans, while smaller pore solid acid catalysts prove highly selective for the dehydrocyclization of xylose to form furfural. In this mechanism, many of the reaction intermediates are of sufficient size that they would be unable to form in the pores of highly acidic smaller pore zeolites (e.g., ZSM-5) but the confines of large pore zeolite acid catalysts (e.g., Beta, faujasite, or mordenite) or mesoporous acid catalysts (e.g., silicate SBA-15 that has been treated with sulfonic or heteropoly acid groups) are ideal for this type of reaction.

The xylans derived from black liquor will degrade more readily than tradition hemicellulose fractions due to the increased processing experienced by these feed stocks. The conversion of xylose to furfural may be.initially be catalyzed by solid acid catalysts alone, but it should be understood that using a process that combines homogeneous and heterogeneous catalysts may be used. Products yields for separate, as well mixed xylan and xylose feeds, that have been exposed to dissolved carbon dioxide (a weak acid) and solid acid catalysts (strong acids) may show increased reaction rates and yields for the production of saccharides or furfural, so as to reduce reactor residence times. For the dehydrocyclization reactions, the reduction of the reactor residence enables the use of smaller reactors and possibly provides added benefits in the form of lower amounts of condensation reaction byproducts being formed. The concentration of the furfural in the reaction mixture depends on the concentration of the hemicellulose following the treating step.

As stated above, the furfural is at a concentration below what is desirable for commercial production. Thus, it is normally desirable to separate and recover the furfural from the water and other impurities in the mixture. This separation may be done, either by way of distillation or extraction. When using a distillation system, the first stage may be a steam stripping column to take advantage of the azeotrope, taking the furfural-water azetrope overhead, condensing and cooling to yield a furfural-rich phase at about 95% furfural and refluxing the water-rich phase, which is depleted in furfural. Then, distillation in a second column produces an even purer bottoms furfural product, which can, if desired, be further purified by distillation, adsorption, or other known methods. Alternatively, carrying out an extraction prior to distillation separates the substantial levels (>80%) of water from the organics so that the distillation can work more efficiently—especially related to energy—since water is removed before distillation. Methylisobutyl ketone (MIBK) is a good solvent since the selectively allows only 1:10 ratio of MIBK:reaction mixture. It will be understood by those skilled in the art that other solvents with similar polarity, solubility, and volatility also are suitable. The MIBK:furfural mixture would then be removed in a much smaller distillation column to separate the MIBK to be recycles as a distillate, and the pure furfural removed as a bottom stream. The selection method of separating the furfural from water is determined based on yield, product purity, and economics. The furfural/water mixture represents a separations challenge because of the low solids content.

The benefits to making furfuraLusing-this process include, among others, a straightforward process that follows one of the several lignin recovery processes currently used or under development, requiring relatively small incremental capital investment, leveraging existing technologies (ultrafiltration, catalysis, and distillation) in a logical sequence to provide a new process to produce furfural, and low cost of operation, since the feedstock is valued only for its fuel value, and the consumed raw materials are essentially zero when heterogeneous catalysts are be used.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A process for the production of furfural from black liquor comprising:
(a) removing lignin from said black liquor of the kraft pulping process by carbonizing said black liquor from the kraft pulping process to insolubilize lignin, neutralize NaOH and other components contained therein and provide a black liquor solution containing hemicellulose;
(b) treating said carbonized black liquor to remove high molecular weight lignin and inorganic salts and concentrate the remaining hemicellulose-containing mixture; and
(c) catalyzing the xylans in said hemicellulose-containing mixture for a sufficient time to convert said xylans to a furfural-containing mixture.

2. The process according to claim 1 further comprising purifying said furfural-containing mixture to separate furfural from the water and other impurities.

3. The process according to claim 1 wherein said carbonizing of said black liquor is carried out by contacting said black liquor with carbon dioxide in an amount sufficient to reduce the pH to less than pH 10.

4. The process according to claim 1 wherein said carbonizing of said black liquor is carried out by contacting said black liquor with carbon dioxide in an amount sufficient to reduce the pH to between a pH of 8.5 and 9.5.

5. The process according to claim 1 wherein said carbonizing is carried out at a temperature between about 60° C. and about 150° C.

6. The process according to claim 1 wherein said carbonizing is carried out at a temperature between about 90° C. and about 130° C.

7. The process according to claim 1 wherein said treating step is two sequential membrane separations wherein the first membrane is ultrafiltration and said second filtration is nanofiltration.

8. The process according to claim 1 wherein said treating step is two sequential separations wherein the first separation is via centrifuge or dissolved-air floatation to separate the high molecular weight components.

9. The process according to claim 1 wherein said catalyst is from the group consisting of amorphous sulfated zirconia and tungstosilicic acid.

10. The process according to claim 1 wherein said catalyst is a zeolite catalyst.

11. The process according to claim 1 wherein said reacting step comprises two separate catalysts wherein said xylans in said hemicellulose-containing mixture are converted to pentose and then the pentose converted to furfural at a concentration of about 5% to about 40%.

12. The process according to claim 2 wherein said purifying step is a distillation of the furfural-containing mixture from said reacting step.

13. A process for the production of furfural from black liquor comprising:
(a) removing lignin from said black liquor of the haft pulping process by carbonizing said black liquor from the kraft pulping process with carbon dioxide in an amount sufficient to reduce the pH to less than pH 10 at a temperature between about 60° C. and 150° C. to insolubilize lignin, neutralize NaOH and other components contained therein and provide a black liquor solution containing hemicellulose;
(b) filtering said carbonized black liquor to remove inorganic salts and concentrate the remaining hemicellulose-containing mixture;
(c) catalyzing the xylans in said hemicellulose solution with a catalyst for a sufficient time to convert said xylans to a furfural-containing mixture; and
(d) purifying said mixture containing furfural to separate furfural.

14. The process according to claim 13 wherein said carbonizing of said black liquor is carried out by contacting said black liquor with carbon dioxide in an amount sufficient to reduce the pH to between a pH of 8.5 and 9.5.

15. The process according to claim 13 wherein said carbonizing step is carried out at a temperature between about 90° C. and about 130° C.

16. The process according to claim 13 wherein said filtering step is a two sequential membrane separation wherein said first membrane is ultrafiltration and said second filtration is nanofiltration.
